(19) United States
(12) Patent Application Publication
(10) Pub. No.: US 2025/0312562 A1
(43) Pub. Date: Oct. 9, 2025

(54) USER STATE INFORMATION-BASED SLEEP INDUCING DEVICE AND METHOD

(71) Applicant: BRLAB INC., Seoul (KR)

(72) Inventors: Jongmin LEE, Seoul (KR); Hyunbin KWON, Seoul (KR); Kwangsuk PARK, Seoul (KR); Byunghun CHOI, Seoul (KR); Jonghyeok JEONG, Seoul (KR)

(21) Appl. No.: 19/245,079

(22) Filed: Jun. 20, 2025

Related U.S. Application Data
(63) Continuation of application No. PCT/KR2023/021428, filed on Dec. 22, 2023.

(30) Foreign Application Priority Data
Dec. 22, 2022 (KR) 10-2022-0182003
Dec. 22, 2022 (KR) 10-2022-0182004
Apr. 10, 2023 (KR) 10-2023-0047032
Nov. 3, 2023 (KR) 10-2023-0151053

(51) Int. Cl. A61M 21/02; G16H 40/60

(52) U.S. Cl. CPC A61M 21/02; G16H 40/60; A61M 2205/3303

(57) ABSTRACT

The present disclosure relates to a user state information-based sleep inducing device and method, and more particularly, the user state information-based sleep inducing device according to the present disclosure may include a layer unit having a plurality of components; a detection unit provided in the layer unit to detect user state information at least twice; a memory unit in which user state information detected by the detection unit is updated and stored; a control unit that generates a sleep inducing signal according to user state information updated and stored in the memory unit; and a stimulation generation unit that generates sleep inducing stimulation according to a signal generated from the control unit.

### CROSS-REFERENCE TO RELATED APPLICATIONS

This application is a Continuation of Application No. PCT/KR2023/021428, filed on Dec. 22, 2023, which in turn claims the benefit of Korean Patent Applications No. 10-2022-0182003, filed on Dec. 22, 2022, No. 10-2022-0182004, filed on Dec. 22, 2022, No. 10-2023-0047032, filed on Apr. 10, 2023, and No. 10-2023-0151053, filed on Nov. 3, 2023. The entire disclosures of all these applications are hereby incorporated by reference.

### TECHNICAL FIELD

The present disclosure relates to a user state information-based sleep inducing device and method, and more particularly, to a user state information-based sleep inducing device and method that monitors a change in user state information in response to stimulation and generates a sleep inducing signal by reflecting the change.

### BACKGROUND ART

Recently, as people's living standards and quality have improved, the demand for ‘good sleep’ has increased, and in particular, the industry that provides various sleep inducing devices or services using the latest scientific technologies is growing significantly. Accordingly, many devices and services are being commercialized to improve sleep quality, such as sleep care services that provide guidance on sleep environment, habits, and posture through consulting with experts, and services that monitor a sleep state by detecting a user's breathing sounds, or the like when wearing a wearable device.

Specifically, various devices and services are being developed that receive information such as the user's physical information, state information, and sleep habits, or collect information such as the user's state information or sleep habits through individual devices that are connected to the user in a contact or non-contact manner, and function to induce ‘good sleep’ based on such information on the user, thereby inducing a sleep state based on user-specific state information.

Illustratively, Korean Patent Publication No. 10-2268804 relates to a device and method of providing lighting and music according to a sleep state using AI, and discloses a configuration that selects lighting and music to induce a user's final sleep state based on the user's biometric information measured through a wearable device. In addition, many technologies have been disclosed to induce the user's sleep state based on user information measured in various ways.

However, in the case of prior arts, there is disclosed only a method of collecting user information in various ways and setting an environment for inducing a sleep state based thereon, and there is a disadvantage in that it is difficult to respond flexibly when the collected user information changes.

The present disclosure has been proposed to complement the disadvantage of the prior art, and provides a user state information-based sleep inducing device and method, which can continuously collect and monitor user information and reflect the same to provide an optimal sleep state for each user, instead of simply inducing a sleep state based on user information collected one-time.

### DISCLOSURE OF INVENTION

### Technical Problem

An aspect of the present disclosure is to provide a user state information-based sleep inducing device and method that can frequently detect and collect user state information, thereby generating and providing a sleep inducing signal reflecting current user state information.

In addition, an aspect of the present disclosure is to provide a user state information-based sleep inducing device and method that can monitor a result of state change in response to sleep inducing stimulation provided to a user and generate a sleep inducing signal based on the monitored result value, thereby generating and providing a more effective sleep inducing signal to the user.

Moreover, an aspect of the present disclosure is to provide a user state information-based sleep inducing device and method that can repeatedly perform a process of detecting state information and generating a sleep inducing signal until the user's state information is determined to be a sleep state, thereby finally inducing a user's sleep state.

Meanwhile, technical problems of the present disclosure are not limited to the above-mentioned problems, and other technical problems which are not mentioned herein will be clearly understood by those skilled in the art from the description below.

### Technical Solution

A user state information-based sleep inducing device according to the present disclosure may include a layer unit having a plurality of components; a detection unit provided in the layer unit to detect user state information at least twice; a memory unit in which user state information detected by the detection unit is updated and stored; a control unit that generates a sleep inducing signal according to user state information updated and stored in the memory unit; and a stimulation generation unit that generates sleep inducing stimulation according to a signal generated from the control unit.

In addition, in the user state information-based sleep inducing device according to the present disclosure, the detection unit may include at least one of a pressure sensor, a vibration sensor, a position sensor, an acoustic sensor, an infrared sensor, a motion sensor, and a facial recognition sensor.

In addition, in the user state information-based sleep inducing device according to the present disclosure, the detection unit may detect user state information prior to and subsequent to generating stimulation from the stimulation generation unit.

In addition, in the user state information-based sleep inducing device according to the present disclosure, the memory unit may accumulate and update user state information detected by the detection unit without deleting the user state information.

In addition, in the user state information-based sleep inducing device according to the present disclosure, the control unit may adjust at least one of an interval, an intensity, a duration, and an intensity by time zone of a sleep inducing signal according to user state information updated and stored in the memory unit.

In addition, in the user state information-based sleep inducing device according to the present disclosure, the control unit may change and generate a sleep inducing signal when the user state information updated and stored in the memory unit exceeds or falls below a predetermined reference value.

In addition, in the user state information-based sleep inducing device according to the present disclosure, the control unit may not generate a sleep inducing signal when user state information updated and stored in the memory unit is determined to be in a sleep state.

A user state information-based sleep inducing method according to the present disclosure, which is performed in a sleep inducing device including a layer unit, a detection unit, a memory unit, a control unit, and a stimulation generation unit, may include detecting user state information for a first time from a detection unit provided in the layer unit; storing user state information detected for the first time by the detection unit in the memory unit; generating a first sleep inducing signal by the control unit according to user state information stored in the memory unit; generating sleep inducing stimulation from a stimulation generation unit according to the first sleep inducing signal generated from the control unit; detecting user state information for a second time subsequent to generating the sleep inducing stimulation from the detection unit; updating and storing the user state information detected for the second time by the detection unit in the memory unit; and generating a second sleep inducing signal according to the user state information updated and stored in the memory unit from the control unit.

In addition, in the user state information-based sleep inducing device according to the present disclosure, the updating and storing of the user state information detected for the second time by the detection unit in the memory unit may include accumulating and updating the user state information detected for the first time by the detection unit without being deleted.

In addition, in the user state information-based sleep inducing method according to the present disclosure, the generating, by the control unit, a primary sleep inducing signal may be performed to adjust at least one of an interval, an intensity, a duration, and an intensity by time zone of the sleep inducing signal according to user state information stored in the memory unit.

In addition, in the user state information-based sleep inducing device according to the present disclosure, the generating a second sleep inducing signal from the control unit may include changing and generating, when the user state information updated and stored in the memory unit exceeds or falls below a predetermined reference value, at least one of an interval, an intensity, a duration, and an intensity by time zone of the first sleep inducing signal.

In addition, in the user state information-based sleep inducing device according to the present disclosure, the method may further include, subsequent to generating a second sleep inducing signal from the control unit, generating sleep inducing stimulation from a stimulation generation unit according to a second sleep inducing signal generated from the control unit.

In addition, in the user state information-based sleep inducing device according to the present disclosure, the method may further include, repeatedly performing n times, subsequent to generating sleep inducing stimulation from a stimulation generation unit according to the second sleep inducing signal generated from the control unit, (n-1) detecting user state information for an n-th time subsequent to generating the sleep inducing stimulation from the detection unit; (n-2) updating and storing the user state information detected for the n-th time from the detection unit in the memory unit; (n-3) generating an n-th sleep inducing signal according to the user state information updated and stored in the memory unit from the control unit; and (n-4) generating sleep inducing stimulation from a stimulation generation unit according to the n-th sleep inducing signal generated from the control unit.

In addition, in the user state information-based sleep inducing method according to the present disclosure, when user state information detected for an n-th time by the control unit is determined to be in a sleep state, the repeated execution of steps (n-1) to (n-4) may be stopped.

### Advantageous Effects

According to the present disclosure, user state information may be frequently detected and collected, and a sleep inducing signal may be generated and sleep inducing stimulation may be provided by reflecting the detected current user state information, thereby more effectively inducing a sleep state for a user.

In addition, according to the present disclosure, a result of state change for sleep inducing stimulation provided to the user may be monitored, and an interval, an intensity, a time, and the like of a sleep induction signal may be adjusted and changed based on the monitored result value, thereby generating and providing a more effective and optimal sleep inducing signal to the user.

Moreover, according to the present disclosure, instantaneous monitoring whether sleep inducing stimulation provided to the user is actually appropriate for inducing a sleep state may be allowed, and a sleep-inducing signal may be adjusted, changed and generated based on current user state information by reflecting the monitoring, thereby shortening a time taken for the user to be induced into a sleep state.

Furthermore, according to the present disclosure, a process of detecting state information and generating a sleep inducing signal until the user's state information is determined to be a sleep state may be repeatedly performed, thereby continuously generating the sleep inducing signal and providing sleep inducing stimulation until the user's sleep state is finally reached.

Meanwhile, the effects of the present disclosure may not be limited to the above-mentioned effects, and other technical effects which are not mentioned herein will be clearly understood by those skilled in the art from the description below.

### BRIEF DESCRIPTION OF THE DRAWINGS

FIG. 1 is a diagram for explaining a user state information-based sleep inducing device according to an embodiment of the present disclosure.

FIG. 2 is a diagram for explaining user state information stored in a memory unit of a user state information-based sleep inducing device in the present disclosure.

FIG. 3 is a flowchart for explaining a user state information-based sleep inducing method according to an embodiment of the present disclosure.

FIG. 4 is a flowchart for explaining a user state information-based sleep inducing method according to another embodiment of the present disclosure.

FIGS. 5A and 5B are diagrams for explaining a method for generating a user state information-based sleep inducing signal according to an embodiment of the present disclosure.

FIGS. 6A and 6B are diagrams for explaining a method for generating a user state information-based sleep inducing signal according to another embodiment of the present disclosure.

FIG. 7 shows how a sleep inducing device operates according to still another embodiment of the present disclosure.

### BEST MODE FOR CARRYING OUT THE INVENTION

The details of the objects and technical configurations of the present disclosure and operational effects thereof will be more clearly understood from the following detailed description based on the accompanying drawings appended hereto. Hereinafter, embodiments according to the present disclosure will be described in detail with reference to the accompanying drawings.

Embodiments disclosed herein should not be interpreted as limiting or used to limit the scope of the present disclosure. It is apparent for those skilled in the art that a description including embodiments herein has various applications. Therefore, any embodiments described in the detailed description of the present disclosure are illustrative for better understanding of the present disclosure and are not intended to limit the scope of the present disclosure to the embodiments.

Functional blocks illustrated in the drawings and described hereunder are only examples of possible implementations. In other implementations, other functional blocks may be used without departing from the concept and scope of the detailed description. Furthermore, one or more functional blocks of the present disclosure are illustrated as separate blocks, but one or more of the functional blocks of the present disclosure may be a combination of various hardware and software elements that execute the same function.

In addition, an expression that some elements are “included” is an expression of an “open type”, and the expression simply denotes that the elements are present, but should not be construed as excluding additional elements. Moreover, in case where it is mentioned that one element is “connected” or “coupled” to the other element, it should be understood that one element may be directly connected to the other element, but another element may be present therebetween.

FIG. 1 is a diagram for explaining a user state information-based sleep inducing device according to the present disclosure.

Referring to FIG. 1, a user state information-based sleep inducing device 100 according to the present disclosure includes at least one layer unit 110 having a plurality of components, a detection unit 120 provided in the layer unit 110 to detect user state information, a memory unit 130 in which the user state information detected by the detection unit 120 is stored, a control unit 140 that generates a sleep inducing signal according to the user state information stored in the memory unit 130, and a stimulation generation unit 150 that generates sleep inducing stimulation according to the signal generated from the control unit 140.

The layer unit 110 may be understood as a member having a receiving space in which the components to be described later can be arranged, and if a receiving space is provided, there is no limitation on the material or shape of the layer unit 110. The layer unit 110 may be a space where a user sleeps, for example, a mattress, or may be any one of a plurality of surfaces constituting a mattress. Additionally, the layer unit 110 may be a mat that may be placed on a mattress, and may furthermore be a member made of wood or metal rather than cotton with a fiber material. In this manner, if the layer unit 110 has a predetermined receiving space, there is no limitation on the material or shape. However, in order to help understand the disclosure, in this detailed description, the explanation will be continued assuming that the layer unit 110 is a mattress.

The detection unit 120, which is provided on the layer unit 110, is configured to detect and acquire state information from a user. The detection unit 120 may include various types of sensors that can detect user state information, such as a pressure sensor, a vibration sensor, a position sensor, an acoustic sensor, an infrared sensor, a motion sensor, a facial recognition sensor, and the like, and may be configured with a combination of at least one or more of the sensors.

Specifically, the detection unit 120 may detect the user's weight or position information through a pressure sensor, or detect the user's vibration signal through a vibration sensor to acquire state information such as a heart rate, a respiratory state, and a movement state. In addition, the detection unit 120 may recognize the user's voice through an acoustic sensor, and may detect the user's gesture through a motion sensor to receive feedback information.

In this way, the detection unit 120 of the present disclosure may be a component for detecting various state information of a user located on the layer unit 110, and adjusting the type, number, layout, and the like of sensors used to detect more precise and specific user state information cannot be limited to an embodiment of the present disclosure.

In addition, the detection unit 120 may detect the user's state information located on the layer unit 110 at least twice. Specifically, the detection unit 120 may detect user state information prior to and subsequent to generating stimulation from the stimulation generation unit 150.

This is to apply a closed loop control method, which is one of system and device control methods, to the sleep inducing device 100 of the present disclosure, and generate a sleep inducing signal in the control unit 140 by reflecting a result value of stimulation generated according to a sleep inducing signal, that is, user feedback information on sleep inducing stimulation so as to reduce an error range with respect to a target value for inducing the user's sleep.

To this end, the present disclosure does not acquire user state information once (in the case of prior arts, once for the first time before the user falls asleep) and generate a sleep inducing signal suitable for the user based thereon, but acquires user state information even after stimulation is generated from the stimulation generation unit 150 and generates a sleep inducing signal reflecting the user state information from the control unit 140, and therefore, the detection unit 120 detects user state information at least twice, for example, prior to and subsequent to generating stimulation from the stimulation generation unit 150.

The memory unit 130, which is a component in which user state information detected by the detection unit 120 is stored, may be provided by being built into the sleep inducing device 100 or may be an external device connected to the sleep inducing device 100 through a wired or wireless communication manner.

Here, the memory unit 130 may store the detected user state information, and the user state information detected at least twice by the detection unit 120 may be updated and stored. That is, the memory unit 130 updates and stores the information value whenever the user state information is updated in the detection unit 120, but previously detected user state information may be accumulated and updated without being deleted, thereby making it possible to continuously monitor the user's state information value.

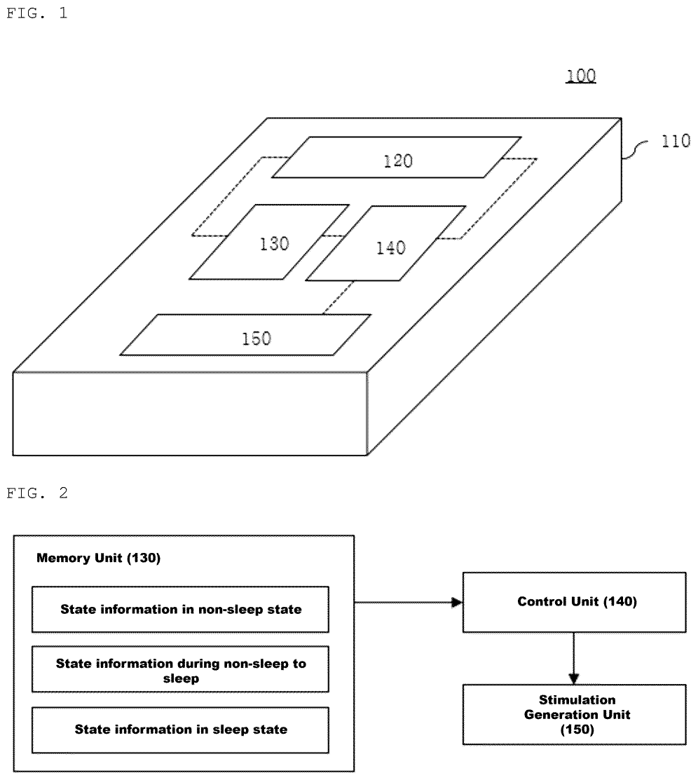

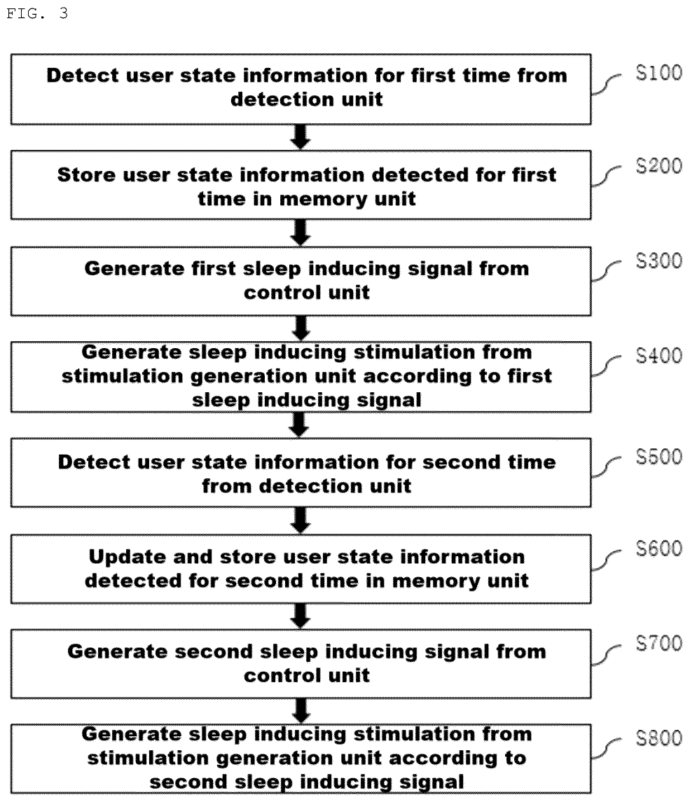

In addition, since the user's state information is continuously monitored and stored in the memory unit 130, the information may be stored separately by the user's sleep or non-sleep state. FIG. 2 is a diagram for explaining user state information stored in a memory unit of a user state information-based sleep inducing device of the present disclosure, and as shown in FIG. 2, the memory unit 130 may periodically collect biometric information values such as heart rate information and breathing cycle of a specific user, and separately store state information in a non-sleep state, state information during non-sleep to sleep, state information in a sleep state, and the like of the user, and the control unit 140 may generate and change a sleep inducing signal according to the information for each user state distinguished in the memory unit 130.

The control unit 140 is configured to generate a sleep inducing signal that induces the user to sleep according to the user state information stored in the memory unit 130. For reference, the control unit 140 may also be understood as a central processing unit. The central processing unit may also be referred to as a controller, a microcontroller, a microprocessor, a microcomputer, or the like. Furthermore, the central processing unit may be implemented by hardware or firmware, software, or a combination thereof, and configured to include an application specific integrated circuit (ASIC) or a digital signal processor (DSP), a digital signal processing device (DSPD), a programmable logic device (PLD), or a field programmable gate array (FPGA) when implemented using hardware, and configured with firmware or software to include a module, a procedure, a function or the like that performs the foregoing functions or operations when implemented using firmware or software.

Here, the control unit 140 according to an embodiment of the present disclosure may generate a sleep inducing signal according to the user state information updated and stored in the memory unit 130. That is, the control unit 140 may adjust and generate at least one of an interval, an intensity, a duration, and an intensity by time zone of a sleep inducing signal according to the user state information stored in the memory unit 130, and in particular, change and generate a sleep inducing signal when the user state information updated and stored in the memory unit 130 exceeds or falls below a predetermined reference value, and may not generate a sleep inducing signal when the user state information updated and stored in the memory unit 130 is determined to be in a sleep state.

Through this, the control unit 140 of the present disclosure may adjust and generate a sleep inducing signal by reflecting the user state information updated and stored in the memory unit 130, thereby generating the sleep inducing signal in a manner of reducing an error between a target value that induces the user into a sleep state and a current user state information value.

The stimulation generation unit 150, which is a component that generates sleep inducing stimulation according to a signal generated from the control unit 140, may include a vibration generation module or a sound generation module. Meanwhile, the vibration generation module or sound generation module may be driven to generate vibration stimulation or sound stimulation using a principle similar to that of a woofer speaker, and may be driven to output vibration stimulation and sound stimulation simultaneously when a stronger stimulation is desired. That is, the stimulation generation unit 150 of the present disclosure may induce the user's sleep in a manner suitable for the user or preferred by the user among vibration stimulation, sound stimulation, or vibration and sound stimulation.

As described above, in the sleep inducing device 100 according to the present disclosure, as the user state information is detected at least twice by the detection unit 120 and updated and stored in the memory unit 130, the user status information may not only be stored separately by sleep/non-sleep state, but also be continuously monitored and stored, and the control unit 140 may adjust and generate an interval, an intensity, a time, and the like of a sleep inducing signal according to the user status information updated and stored in the memory unit 130 so as to generate a sleep inducing signal that is most appropriate for current user status information.

That is, the sleep inducing device 100 according to the present disclosure may not be controlled in a manner of unilaterally providing the same sleep inducing signal and stimulation to the user, but continuously receive feedback on user state information according to a result of the sleep inducing stimulation provided to the user, and provide the user with a sleep inducing signal and stimulation that is adjusted/changed by reflecting the fed-back state information value, thereby gradually reducing an error with respect to a state information target value corresponding to an actual sleep state of the user so as to obtain an effect of providing an optimal sleep state.

FIG. 3 is a flowchart for explaining a user state information-based sleep inducing method according to an embodiment of the present disclosure.

Referring to FIG. 3, the present disclosure, which is a sleep inducing method performed in a sleep inducing device including a layer unit, a detection unit, a memory unit, a control unit, and a stimulation generation unit, may include detecting user state information for a first time from a detection unit provided in the layer unit (S100), storing user state information detected for the first time by the detection unit in the memory unit (S200), generating a first sleep inducing signal by the control unit according to user state information stored in the memory unit (S300), generating sleep inducing stimulation from a stimulation generation unit according to the first sleep inducing signal generated from the control unit (S400), detecting user state information for a second time subsequent to generating the sleep inducing stimulation from the detection unit (S500), updating and storing the user state information detected for the second time by the detection unit in the memory unit (S600), generating a second sleep inducing signal according to the user state information updated and stored in the memory unit from the control unit (S700), and generating sleep inducing stimulation from the stimulation generation unit according to the second sleep inducing signal generated from the control unit (S800).

The configuration in which the user's state information is detected at least twice in the detection unit of the present disclosure and the detected user state information is updated and stored in the memory unit is similar to the content described above in FIGS. 1 and 2, and therefore, the sleep inducing method of the present disclosure controlled in a closed loop manner will be described in more detail below.

First, the detecting of user state information for a first time from a detection unit provided in the layer unit (S100) is performed to detect at least one or more information items among the user's weight, height, body proportions, identification information, and sleep state through a detection unit including at least one or more sensors among a pressure sensor, a vibration sensor, a position sensor, an acoustic sensor, an infrared sensor, a motion sensor, and a facial recognition sensor. The detection unit may detect not only the user's physical information, but also user-specific identification information such as a face or a voice, and biometric information such as a heart rate and a breathing cycle.

Next, the storing of user state information detected for the first time by the detection unit in the memory unit (S200) is performed. In step S200, the user state information detected for a first time in the detection unit may be mapped and stored for each user, and information on the user's sleep or non-sleep state may be stored separately.

Next, the generating of a first sleep inducing signal by the control unit according to user state information stored in the memory unit (S300) is performed. Specifically, the control unit may generate a sleep inducing signal so as to allow the user state information stored in the memory unit to reach a target value corresponding to a sleep state, and for example, generate at least one of an interval, an intensity, a duration, and an intensity by time zone of the sleep inducing signal to be adjusted.

Next, the generating of sleep inducing stimulation from a stimulation generation unit according to the first sleep inducing signal generated from the control unit (S400) is performed. The stimulation generation unit may include at least one of a vibration generating module and a sound generating module, and the generating of sleep inducing stimulation from the stimulation generation unit (S400) may be performed to output vibration or sound in the most optimal manner according to user-specific state information and provided to the user.

Then, the detecting of user state information for a second time subsequent to generating the sleep inducing stimulation from the detection unit (S500) is performed. That is, the present disclosure may apply a closed loop manner that adjusts and provides a sleep inducing signal by reflecting user feedback information rather than continuously providing the user with the same sleep inducing signal once generated, and to this end, step S500 of detecting user state information for a second time as a result value for the first sleep inducing signal is performed.

Next, the updating and storing of the user state information detected for the second time by the detection unit in the memory unit (S600) is performed. The step S600 may be performed so as to allow the information value to be updated and stored whenever the user state information is updated, but the user state information stored and detected for a first time in the step S200 may be accumulated and updated without being deleted, thereby continuously monitoring and collecting the user's state information value. Accordingly, the present disclosure may not only allow the user's state information, which is a result value of sleep inducing stimulation provided from a stimulation generation unit, to be updated to a current value and stored, but also separately collect how biometric information such as a heart rate and a breathing cycle changes depending on the user's non-sleep state, sleep state, or non-sleep to sleep state.

Then, the generating of a second sleep inducing signal according to the user state information updated and stored in the memory unit from the control unit (S700) is performed. In this case, when the user state information updated and stored in the memory exceeds or falls below a predetermined reference value, the control unit generates a second sleep inducing signal in which at least one of an interval, an intensity, a duration, and an intensity by time zone of the primary sleep inducing signal is changed.

That is, the present disclosure may generate a sleep inducing signal that can induce the user's sleep state by using state information corresponding to the user's sleep state as a predetermined target value, and detect the user state information for a second time as a result value of the sleep inducing signal and stimulation provided to the user and compare it with the user state information corresponding to the target value, thereby adjusting and generating a second sleep inducing signal so as to reduce an error from the target value. Through this, the present disclosure may not only more effectively provide a “good sleep” state to the user, but also shorten the time it takes for the user to reach a “good sleep” state.

Next, the generating of sleep inducing stimulation from a stimulation generation unit according to the second sleep inducing signal generated from the control unit (S800) is performed. The step S800 may be performed in a similar manner to the step S400 described above, but may be performed in various ways, such as providing a stronger or weaker intensity for a longer or shorter period of time to induce the user into a sleep state.

Subsequent to step S800, the user may or may not reach the sleep state, which is the goal of the present disclosure. Since the present disclosure aims to induce the user into a state of “good sleep,” a process of detecting user state information and providing sleep inducing stimulation accordingly may be performed repeatedly until the goal is achieved.

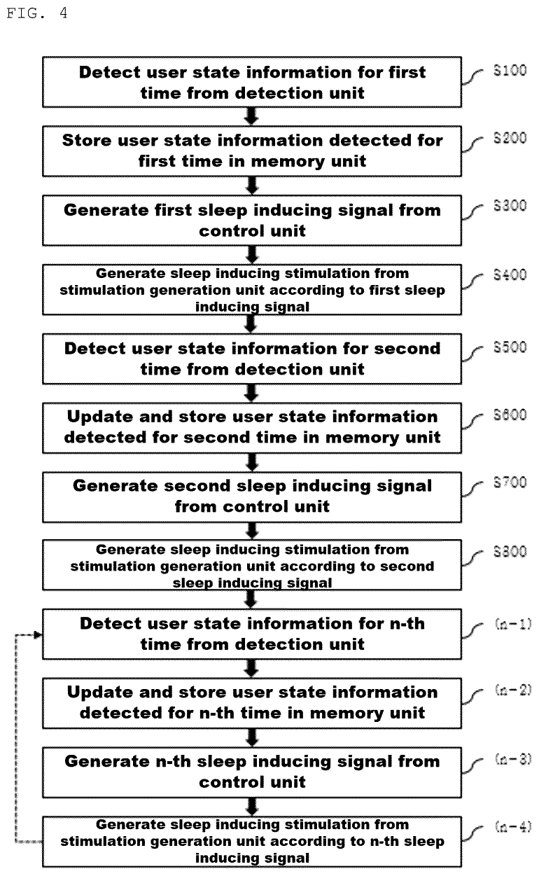

FIG. 4, which is a flowchart for explaining a user state information-based sleep inducing method according to another embodiment of the present disclosure, may include a process in which, when the user has not reached a sleep state even subsequent to step S800, a process of detecting user state information and providing sleep inducing stimulation accordingly is repeated n times.

Hereinafter, a process that can be performed subsequent to step S800 will be described in more detail with reference to FIG. 4.

Subsequent to step S800, the detection unit performs step (n-1) of detecting user state information for an n-th time as a result value of sleep inducing stimulation generated according to the second sleep inducing signal, and then step (n-2) of updating and storing the user state information detected for the n-th time in the memory unit. Then, step (n-3) of generating an n-th sleep inducing signal according to the user state information updated and stored in the memory unit from the control unit is performed, and step (n-4) of generating sleep inducing stimulation from the stimulation generation unit according to the nth sleep inducing signal generated from the control unit is performed.

For reference, the repetition of the steps mentioned in FIG. 4 is not necessarily performed only to induce a user in an awake state (non-asleep state) into a sleep state, and may also be carried out for a user in an asleep state (a sleep state) to induce the user into a specific sleep stage or to cause the user to escape from a specific sleep stage. Sleep may be divided into REM sleep and non-REM sleep, and each non-REM sleep may be further divided into several sleep stages, and the sleep inducing device according to the present disclosure may repeat processes of detecting the user's state information, updating and storing the detected state information, and generating an arbitrary sleep inducing signal to induce the user to change from REM sleep to non-REM sleep, or from a specific stage of non-REM sleep to another stage even when the user is in a sleep state.

Meanwhile, the above steps are not only examples for inducing the user to sleep, but may also be applied when trying to wake the user up. For example, when state information of a pattern of a user waking up from a sleep state is detected, the sleep inducing device may update and store the detected state information and generate stimulation that is adjusted in intensity to avoid discomfort during the user's awakening process, or stimulation with an appropriate cycle to wake the user up (e.g., stimulation with a shorter cycle by a set percentage than the user's heart rate value).

On the other hand, the repeated processes performed subsequent to step S800 of FIG. 4 may be executed to respond to an unexpected change in the user's state. Assuming that the user state information is detected while continuous vibration or sound stimulation is being applied and the user shows state information in a pattern of waking up from sleep, the sleep inducing device may respond to a situation in which the user is waking up from sleep by updating and storing the user state information in the memory and then reducing an intensity of the stimulation, that is, by re-adjusting and generating the sleep inducing signal.

That is, the present disclosure aims to induce the user's state (heart rate, respiration rate, etc.) so as to allow the user to be placed in an optimal sleep environment or optimal wake-up environment, and to this end, the present disclosure may repeatedly perform n times a process of detecting the user's state information and adjusting/changing and generating a sleep inducing signal by reflecting the information. In other words, unlike a prior art that unilaterally provides a sleep inducing signal and stimulation to a user, the present disclosure may repeatedly perform a process of ‘collecting user state information—providing a sleep inducing signal and stimulation based on the collected user state information’ until the user state information reaches a target value, thereby inducing a user sleep state more quickly and effectively.

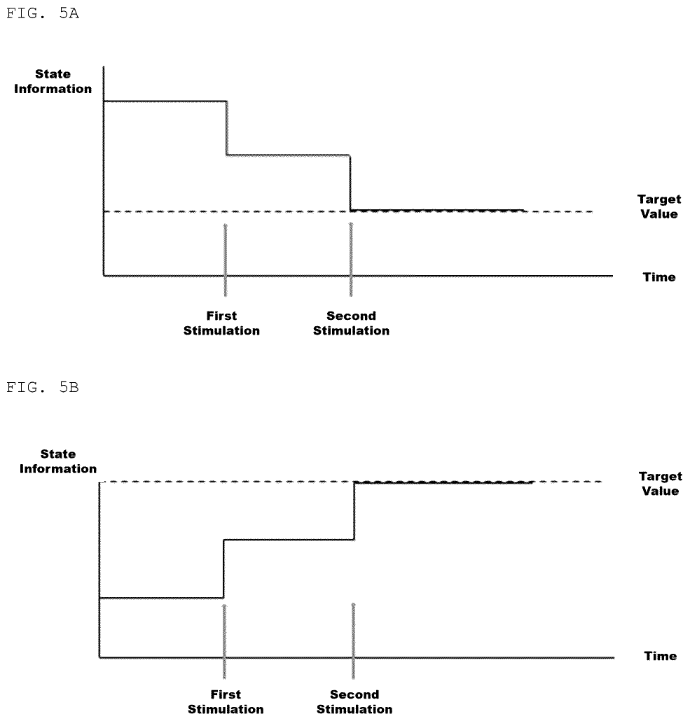

Hereinafter, with reference to FIGS. 5A and 5B, a method of generating a sleep inducing signal according to an embodiment of the present disclosure, which induces a user to sleep until user state information reaches a target value, will be described.

As shown in FIG. 5A, the state information corresponding to the user's sleep state may be set as a target value, and a sleep inducing signal and stimulation may be provided for first and second times until the user's state information decreases to the target value.

Taking the user's heart rate as an example, the sleep inducing device of the present disclosure may continuously detect the user's current heart rate through a detection unit, and may generate a sleep inducing signal for the purpose of lowering the heart rate by a preset target value compared to a current heart rate by setting the heart rate corresponding to the user's sleep state as a target value. When the user's heart rate does not reach the target value even subsequent to generating first stimulation based on the sleep inducing signal, second stimulation may be provided by generating a sleep inducing signal again based on the user's current heart rate, and stimulation may be provided by continuously generating a sleep inducing signal until the user's heart rate reaches the target value (user sleep state).

As shown in FIG. 5B, the state information corresponding to the user's sleep state may be set as a target value, and sleep inducing signal and stimulation may be provided for first and second times until the user's state information increases to the target value.

Taking the user's breathing cycle as an example, the sleep inducing device of the present disclosure may continuously detect the user's current breathing cycle through a detection unit, and may generate a sleep inducing signal for the purpose of setting a breathing cycle corresponding to the user's sleep state as a target value and increasing the breathing cycle by the preset target value compared to the current breathing cycle. The present disclosure may provide second stimulation at the time of inspiration by generating a sleep inducing signal again based on the current user breathing cycle when the user's breathing cycle does not reach the target value even subsequent to generating first stimulation based on a sleep inducing signal, thereby continuously generating a sleep inducing signal and providing stimulation until the user's breathing cycle reaches the target value (user sleep state).

That is, the present disclosure may repeatedly perform a process of setting state information (e.g., heart rate, breathing cycle, etc.) corresponding to a user sleep state as a target value, collecting user state information until the target value is reached, and generating a sleep inducing signal according to the collected state information, and in particular, may generate a sleep inducing signal based on the collected user state information to gradually reduce an error between a target value and current state information so as to reach a final target value. Through this, the present disclosure may provide sleep inducing stimulation in a manner, such as an intensity and a time, that is most appropriate for the user's current state, thereby quickly and effectively providing the user with a “good sleep” state.

Moreover, the present disclosure may also induce a user's sleep by correcting a target value when there is a large error between user state information, which is a result of sleep inducing stimulation provided to the user, and the target value.

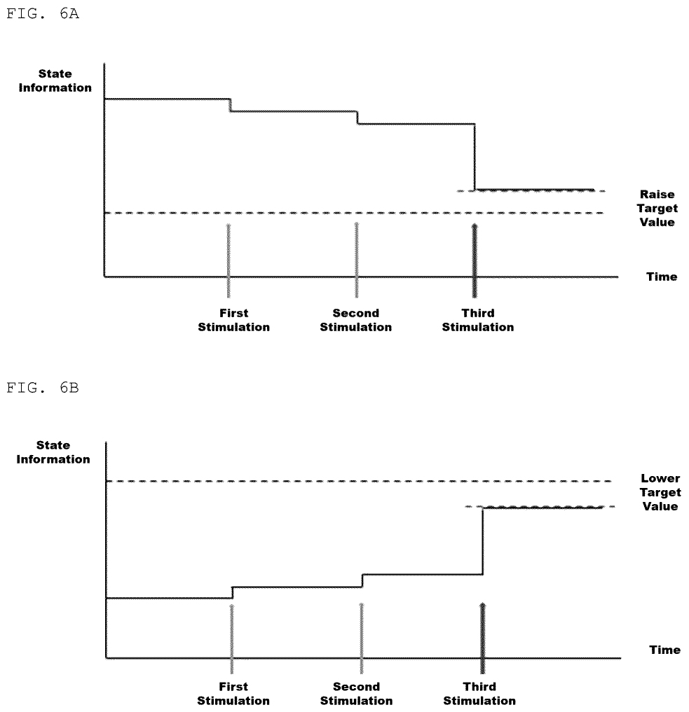

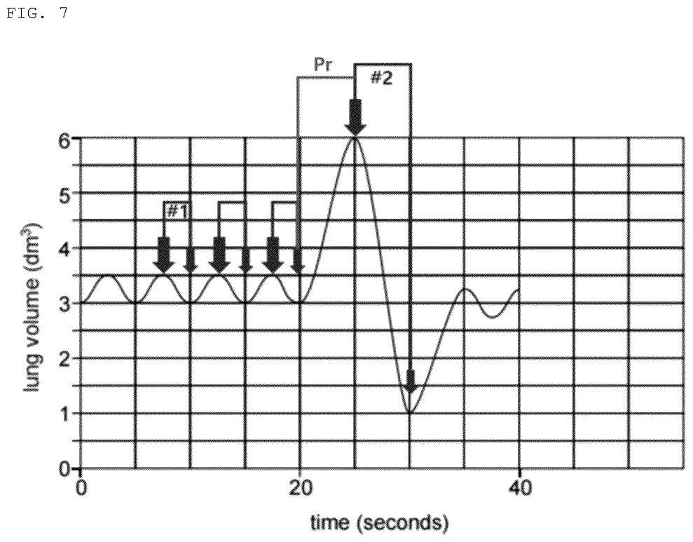

FIGS. 6A and 6B are diagrams for explaining a method for generating a sleep inducing signal based on user state information according to another embodiment of the present disclosure, and referring to FIG. 6A, when there is no significant change in the user state information despite providing the user with the first and second stimulations, a sleep inducing signal may also be generated by raising the set target value as it is considered that the autonomic nervous system is not properly adjusted by the stimulations provided to the user.

Taking the user's heart rate as an example, when there is no significant change in the user's heart rate even subsequent to providing the first and second stimulations so as not to allow an error in the target value to be greatly reduced, the preset target value may be upwardly set to be slightly closer to the user's current heart rate, thereby controlling a sleep inducing signal to be generated. Through this, third stimulation may be provided to induce a slightly smaller upward target value than the preset target value based on the user's current heart rate, thereby inducing a larger change in the user's heart rate.

Conversely, a sleep inducing signal may be generated by downwardly adjusting the target value, wherein taking the user's breathing cycle as an example, as shown in FIG. 6B, when there is no significant change in the user's breathing cycle even subsequent to providing the first and second stimulations so as not to allow an error in the target value to be greatly reduced, the preset target value may be downwardly set to be slightly closer to the user's current breathing cycle, thereby controlling the sleep inducing signal to be generated. Through this, stimulation may be provided to induce a downward target value with a slightly smaller error than the preset target value based on a current breathing cycle of the user's body, thereby inducing a larger change in the user's breathing cycle.

In addition, as user state information is frequently detected and collected, the present disclosure may instantaneously and flexibly change and generate a sleep inducing signal for inducing the user into a sleep state according to the user state information, and although not specifically shown and described, a method of changing/adjusting a sleep inducing signal and stimulation provided to the user according to the user state information collected in real time may be implemented in various ways, and the technical features and embodiments of the present disclosure for this purpose should not be construed as limited to the content shown and described herein.

In this way, as user state information (heart rate, breathing cycle, etc.) is continuously collected even subsequent to providing sleep inducing stimulation to the user, the present disclosure may identify the user state information as a result of the sleep inducing stimulation and also determine whether the stimulation provided to the user has been effective in inducing sleep. Accordingly, the present disclosure may set a target value by upwardly or downwardly adjusting it based on current user state information collected subsequent to generating sleep inducing stimulation, and may generate an optimal sleep inducing signal that can effectively induce a user into a sleep state.

Meanwhile, FIG. 7 shows how a sleep inducing device operates according to still another embodiment of the present disclosure. In the above description of FIGS. 5 and 6, only an embodiment of generating a sleep inducing signal by referring to only either one of the user's heart rate and breathing cycle has been described, but the sleep inducing signal may be generated by referring to both a plurality of state information, that is, the heart rate and breathing cycle.

FIG. 7 shows a stimulation time point at which a sleep inducing signal is applied to a user. Although it is shown in FIGS. 5 and 6 a time point when stimulation should be applied based on whether the user's heart rate or breathing cycle has reached a target value by referring to state information, FIG. 7 shows a time point when stimulation should be applied to the user during the user's breathing cycle, and it should be understood that FIG. 7 is a graph for determining a stimulation time point within the cycle in which the stimulation is applied.

When referring to the diagram, if the user's sleep state is to be induced into a deeper sleep stage, that is, the user's heart rate is to be induced to be slower, the sleep inducing device may be controlled to apply stimulation only during the expiration (exhalation) of the user's breathing cycle. Referring to FIG. 7, the y-axis represents a lung volume, the x-axis represents a time, wherein the lung volume on a vertical axis is merely a reference borrowed to distinguish between expiration and inspiration of breathing, and it is understood that any other reference can be used to identify the user's breathing cycle, for example, values obtained by the sleep inducing device according to the present disclosure using a pressure sensor and/or a vibration sensor, or values calculated by processing those values.

Referring to FIG. 7, it can be seen that the sleep inducing device starts to apply stimulation from a time point when lung capacity increases and then decreases, and stops applying stimulation from a time point when lung capacity increases again. That is, when the user's heart rate is induced to slow down, it is more efficient to apply stimulation to induce a slower heart rate when the parasympathetic nervous system is activated, and to this end, the sleep inducing device according to the present disclosure may be designed so as to allow stimulation to be applied only during expiration, which is more influenced by the parasympathetic nervous system. Conversely, if the goal is to wake up the user, that is, to induce a faster heart rate, the sleep inducing device may be controlled to apply stimulation only during the inspiration (inhalation) of the user's breathing cycle.

Meanwhile, when looking closely at FIG. 7, in another embodiment, the sleep inducing device according to the present disclosure may be designed so as to allow an intensity of the stimulation to be adjusted at a time point when expiration starts and at a time point when expiration ends, and for example, the device may be designed so as to allow relatively larger stimulation to be applied at the time point when expiration starts and relatively smaller stimulation to be applied at the time point when expiration ends, and the intensity of the stimulation may be controlled so as to gradually decrease (decay) between the start and end points. On the other hand, the sleep inducing device according to the present disclosure may also perform a calculation to predict how long expiration is to last from the time point when the expiration starts, and for example, in expiration #1, the sleep inducing device may calculate that the expiration is to last for about 2.5 seconds by referring to the accumulated user's state information, and when it is desired to gradually reduce the intensity of the stimulation, the amount of stimulation reduction may be determined by referring to the expiration duration. In the case of expiration #2, it indicates a different aspect of breathing state compared to expiration #1, that is, the expiration that follows when the inspiration is longer and the breathing volume is greater than before, and when such an irregular breathing state is detected, the sleep inducing device may calculate the time point when the expiration ends and/or the amount of stimulation reduction by increasing the proportion of data acquired in the previous inspiration state. That is, when it is desired to calculate the expiration end time point (and/or amount of stimulation reduction) in expiration #2, the sleep inducing device may further increase a weighting ratio of the user state information in the previous inspiration section, denoted as Pr.

In the above, a user state information-based sleep inducing device and method according to the present disclosure has been examined. Meanwhile, the present disclosure is not limited to the foregoing specific embodiments and application examples, it will be of course understood by those skilled in the art that various modifications may be made without departing from the gist of the present disclosure as defined in the following claims, and it is to be noted that those modifications should not be understood individually from the technical concept and prospect of the present disclosure.

In particular, configurations that implement the technical features of the present disclosure included in the block diagrams and flowcharts shown in the drawings attached to this specification represent logical boundaries between the configurations. However, according to an embodiment of software or hardware, the shown configurations and functions thereof are executed in the form of stand-alone software modules, monolithic software structures, codes, services, and combinations thereof, and the functions may be implemented by being stored in a medium executable on a computer provided with a processor capable of executing the stored program codes, instructions, and the like, and therefore, all of these embodiments should also be regarded as falling within the scope of the present disclosure.

Accordingly, the accompanying drawings and technologies thereof describe the technical characteristics of the present disclosure, but should not be simply inferred unless a specific array of software for implementing such technical characteristics is clearly described otherwise. That is, the aforementioned various embodiments may be present, and may be partially modified while having the same technical features as those of the present disclosure, and thus such modified embodiments should also be regarded as falling within the scope of the present disclosure.

Furthermore, the flowchart describes operations in the drawing in a specific sequence, but has been shown to obtain the most preferred result, and it should not be understood that such operations must be carried out in the specific sequence or sequential sequence shown, or that all shown operations must be carried out. In a specific case, multi-tasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

1. A user state information-based sleep inducing device, the device comprising:
a layer unit having a plurality of components;
a detection unit provided in the layer unit to detect user state information at least twice;
a memory unit in which user state information detected by the detection unit is updated and stored;
a control unit that generates a sleep inducing signal according to user state information updated and stored in the memory unit; and
a stimulation generation unit that generates sleep inducing stimulation according to a signal generated from the control unit.

2. The device of claim 1, wherein the detection unit detects user state information prior to and subsequent to generating stimulation from the stimulation generation unit.

3. The device of claim 1, wherein the memory unit accumulates and updates user state information detected by the detection unit without deleting the user state information.

4. The device of claim 1, wherein the control unit changes and generates a sleep inducing signal when the user state information updated and stored in the memory unit exceeds or falls below a predetermined reference value.

5. A sleep inducing method performed in a sleep inducing device comprising a layer unit, a detection unit, a memory unit, a control unit, and a stimulation generation unit, the method comprising:
detecting user state information for a first time from a detection unit provided in the layer unit;
storing user state information detected for the first time by the detection unit in the memory unit;
generating a first sleep inducing signal by the control unit according to user state information stored in the memory unit;
generating sleep inducing stimulation from a stimulation generation unit according to the first sleep inducing signal generated from the control unit;
detecting user state information for a second time subsequent to generating the sleep inducing stimulation from the detection unit;
updating and storing the user state information detected for the second time by the detection unit in the memory unit; and
generating a second sleep inducing signal according to the user state information updated and stored in the memory unit from the control unit.

6. The method of claim 5, wherein the updating and storing of the user state information detected for the second time by the detection unit in the memory unit comprises:
accumulating and updating the user state information detected for the first time by the detection unit without being deleted.

7. The method of claim 5, wherein the generating a second sleep inducing signal from the control unit comprises:
changing and generating, when the user state information updated and stored in the memory unit exceeds or falls below a predetermined reference value, at least one of an interval, an intensity, a duration, and an intensity by time zone of the first sleep inducing signal.

8. The method of claim 5, further comprising:
subsequent to generating a second sleep inducing signal from the control unit,
generating sleep inducing stimulation from a stimulation generation unit according to a second sleep inducing signal generated from the control unit.

9. The method of claim 8, further comprising:
repeatedly performing n times,
subsequent to generating sleep inducing stimulation from a stimulation generation unit according to the second sleep inducing signal generated from the control unit,
(n-1) detecting user state information for an n-th time subsequent to generating the sleep inducing stimulation from the detection unit;
(n-2) updating and storing the user state information detected for the n-th time from the detection unit in the memory unit;
(n-3) generating an n-th sleep inducing signal according to the user state information updated and stored in the memory unit from the control unit; and
(n-4) generating sleep inducing stimulation from a stimulation generation unit according to the n-th sleep inducing signal generated from the control unit.
